(19) United States
(12) Patent Application Publication
(10) Pub. No.: US 2005/0043418 A1
(43) Pub. Date: Feb. 24, 2005

(54) Gas-to-liquid process

(75) Inventor: Kenneth Agee, Bixby, OK (US)

Correspondence Address: Valerie K. Friedrich, Ph.D.;Jenkens & Gilchrist, PC, Suite 2600, 1401 McKinney, Houston, TX, 77010, US

(73) Assignee: Syntroleum Corporation, Tulsa, OK (US)

(21) Appl. No.: 10/913,892

(22) Filed: Aug. 6, 2004

Related U.S. Application Data
(60) Provisional application No. 60/493,293, filed on Aug. 6, 2003.

(51) Int. Cl. C07C 27/06

(52) U.S. Cl. 518/726

(57) ABSTRACT

A method of economically and efficiently converting natural gas into one or more liquid hydrocarbon products is provided in which the method includes the steps of: (a) building a plant comprising one or more modules for the conversion of natural gas into synthesis gas and the conversion of synthesis gas into heavier hydrocarbons and at least one product module on a transportable platform at a location that facilitates heavy construction; (b) transporting the plant to a location containing sufficient natural gas reserves for operation; (c) installing the plant near the natural gas reserve; and (d) producing intermediate or finished liquid hydrocarbon products.

### CROSS REFERENCE TO RELATED APPLICATIONS

This Application claims priority to application Ser. No. 60/493,293, filed on Aug. 6, 2003 and entitled “Fischer-Tropsch Processes and Products.”

### FEDERALLY SPONSORED RESEARCH

Not applicable.

### REFERENCE TO MICROFICHE APPENDIX

Not applicable.

### FIELD OF THE INVENTION

The invention relates to gas to liquid processes, and more particularly to Fischer-Tropsch processes.

### BACKGROUND OF THE INVENTION

While technological advances within the energy industry have made dramatic improvements in lowering the cost of finding, producing and refining oil, vast quantities of remote and stranded gas still wait to be developed. Gas to liquid (“GTL”) technologies may assist in developing and monetizing these resources. Such GTL technologies are especially critical to offshore applications given that about one-half of the world's stranded gas is located in water.

In conventional GTL processes, synthesis gas is generated from natural gas via partial oxidation with oxygen, requiring an air separation plant to provide the oxygen. In conventional approaches, nitrogen is eliminated from the synthesis gas stream as an unwanted inert. In an air-based system, however, synthesis gas is produced by oxidation of hydrocarbons using air-carried oxygen, rather than separated oxygen. This eliminates the expense, as well as the extra space requirment, of an air separation plant. It thus reduces capital costs, making possible plants with considerably smaller footprints, and also provides for a safer operating environment.

Fischer-Tropsch plants of at least about 50,000 B/d production are generally required in order to lower the capital cost per barrel of daily capacity to an acceptable level. However, such Fischer-Tropsch plants require about 500 Mmcf/d of feed gas, or 5.4 trillion cubic feet over a thirty year period. Only about 2% of the known gas fields outside of North America are of such size.

There remains a need therefore, for a process for converting stranded gas reserves of relatively low capacity, of less than about 2 trillion cubic feet, efficiently and economically into higher value hydrocarbon products. There remains a further need for a process which may be conducted using modular components such that the product slate may be adapted to meet market and local needs and conditions.

### SUMMARY OF THE INVENTION

Certain embodiments of the invention provide method of economically and efficiently converting natural gas into one or more liquid hydrocarbon products comprising the steps of: (a) building a plant comprising one or more modules for the conversion of natural gas into synthesis gas and the conversion of synthesis gas into heavier hydrocarbons and at least one product module on a transportable platform at a location that facilitates heavy construction; (b) transporting the plant to a location containing sufficient natural gas reserves for operation; (c) installing the plant near the natural gas reserve; and (d)producing intermediate or finished liquid hydrocarbon products.

### BRIEF DESCRIPTION OF THE DRAWINGS

FIG. 1 is a schematic diagram showing an embodiment of the invention wherein Wellhead Natural Gas and Fischer-Tropsch synthesis product are blended in the production of LPG products, Naphtha products and transportation fuel products.

FIG. 2 is a schematic diagram showing an alternative embodiment of the invention wherein Wellhead Natural Gas and Fischer-Tropsch synthesis product are co-processed in the production of a transportation fuel.

FIG. 3 is a schematic diagram showing another alternative embodiment of the invention wherein Wellhead Natural Gas, Fischer-Tropsch synthesis product, and imported offsite Natural Gas are co-processed in the production of a transportation fuel.

### DESCRIPTION OF EMBODIMENTS OF THE INVENTION

The term “Cₓ”, where x is a number greater than zero, refers to a hydrocarbon compound having predominantly a carbon number of x. As used herein, the term Cₓ may be modified by reference to a particular species of hydrocarbons, such as, for example, C₅ olefins. In such instance, the term means an olefin stream comprised predominantly of pentenes but which may have impurity amounts, i.e. less than about 10%, of olefins having other carbon numbers such as hexene, heptene, propene, or butene. Similarly, the term “Cₓ+” refers to a stream wherein the hydrocarbons are predominantly those having a hydrocarbon number of x or greater but which may also contain impurity levels of hydrocarbons having a carbon number of less than x. For example, the term C₁₅₊ means hydrocarbons having a carbon number of 15 or greater but which may contain impurity levels of hydrocarbons having carbon numbers of less than 15. The term “Cₓ-C_{y}”, where x and y are numbers greater than zero, refers to a mixture of hydrocarbon compounds wherein the predominant component hydrocarbons, collectively about 90% or greater by weight, have carbon numbers between x and y. For example, the term C₅-C₉ hydrocarbons means a mixture of hydrocarbon compounds which is predominantly comprised of hydrocarbons having carbon numbers between 5 and 9 but may also include impurity level quantities of hydrocarbons having other carbon numbers.

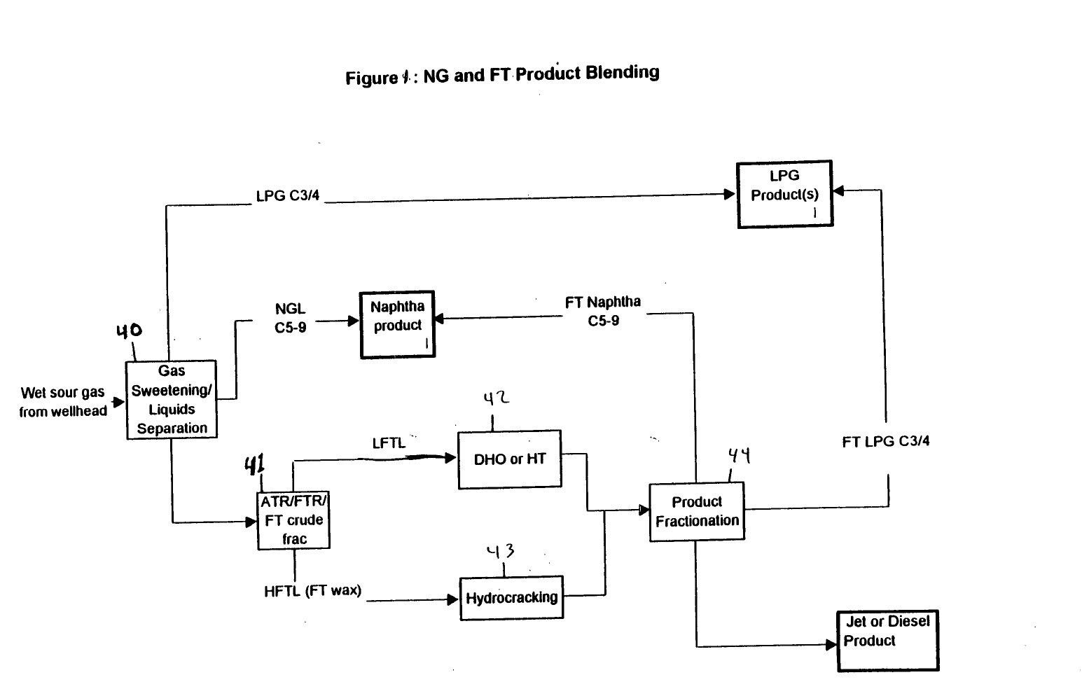

Referring to FIG. 1, another embodiment of the invention is shown in which wet sour gas from a wellhead is processed in a gas sweetening/liquids separation unit 40. An LPG fraction is obtained overhead from unit 40, an NGL fraction is obtained as an upper side stream from unit 40 and a bottoms fraction containing hydrocarbons having a carbon number primarily above 9 is recovered. The bottoms fraction is sent to synthesis gas production, as described above, with the synthesis gas then being used in a Fischer-Tropsch synthesis, also as described above. The Fischer-Tropsch synthesis product is then fractionated to recover a light Fischer-Tropsch liquid, also commonly referred to as a Fischer-Tropsch oil, (“LFTL”) and a heavy Fischer-Tropsch liquid, also commonly referred to as a Fischer-Tropsch wax, (“HFTL”). In FIG. 1, the synthesis gas production, Fischer-Tropsch synthesis and Fischer-Tropsch product fractionation processes are jointly illustrated in unit 41. The Fischer-Tropsch LFTL may then be dehydrated, or otherwise treated for removal of oxygenates, or hydrotreated, as indicated in FIG. 1 in unit 42. The HFTL may be hydrocracked in unit 43 to produce lower molecular weight hydrocarbons. The products of units 42 and 43 may then be recombined and fed into a product fractionator 44. A Fischer-Tropsch LPG product may be recovered from Fractionator 44 and combined with the LPG obtained overhead from unit 40. The combined LPG may then be further processed into a variety of known LPG products. A Fischer-Tropsch Naphtha may also be recovered from fractionator 44 and combined with the NGL recovered from unit 40. The combined NGL and Fischer-Tropsch Naphtha may then be further processed into a variety of known naphtha products. Transportation fuels, or blending stocks therefor, such as jet fuel and diesel fuel may also be recovered from fractionator 44.

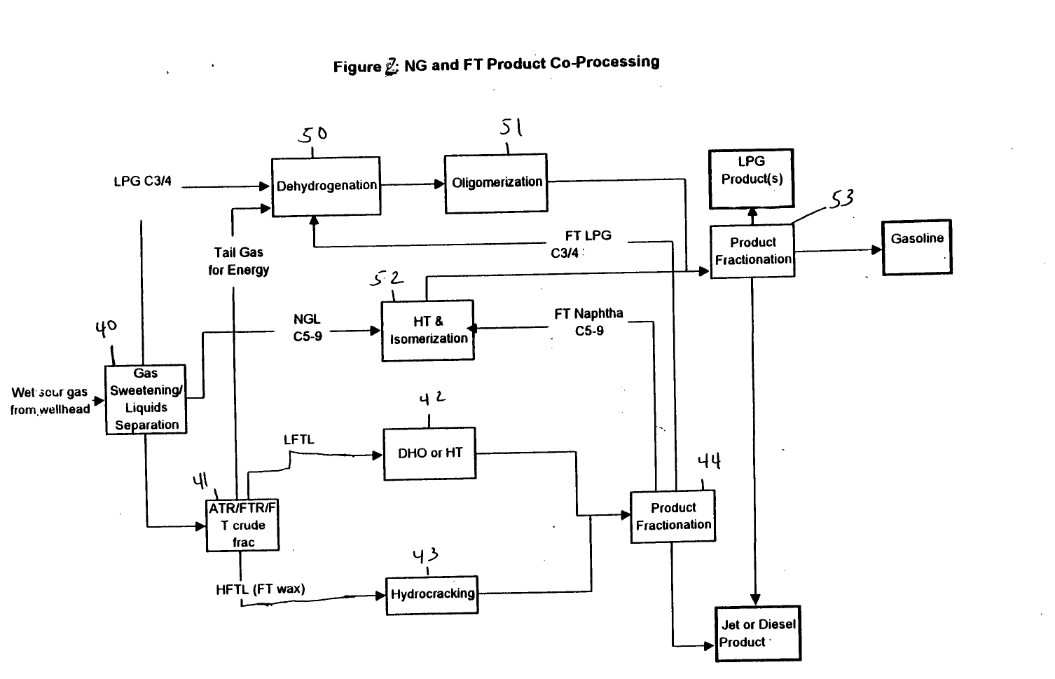

Referring now to FIG. 2, another alternative embodiment of the invention is shown in which the LPG recovered from unit 40 and the Fischer-Tropsch LPG recovered from fractionator 44 are dehydrogenated in dehydrogenator 50. Unsaturated hydrocarbons produced in dehydrogenator 50 are then oligomerized in oligomerization unit 51. NGL from unit 40 and Fischer-Tropsch Naphtha from fractionator 44 are combined and hydrotreated and hydroisomerized in unit 52. The product of unit 52 is then combined with the oligomerization product from unit 51 and the combined hydrocarbon mixture, which contains predominantly hydrocarbons having between about 6 and 10 carbons, are introduced into a second product fractionator 53. An overhead LPG stream, containing saturated hydrocarbons and non-oligomerized unsaturated hydrocarbons may be recovered overhead from fractionator 53. A gasoline fraction and a combined jet and diesel fuel fraction may also be recovered from fractionator 53. The jet and diesel fractions from fractionators 53 and 44 may be combined for further processing or storage.

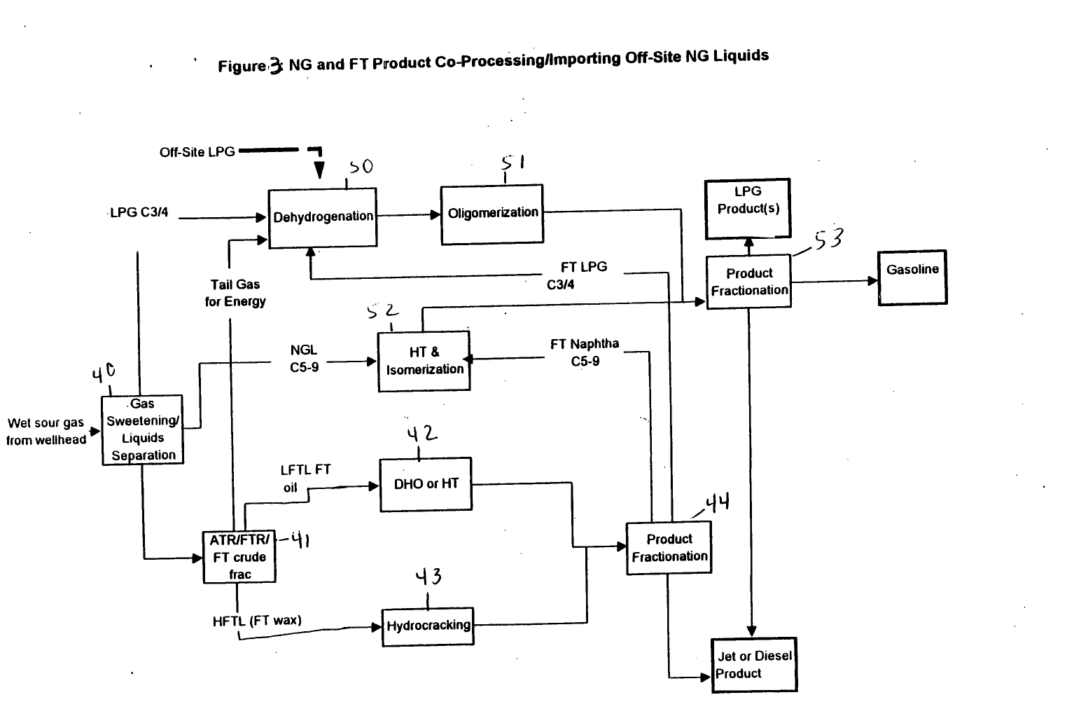

FIG. 3 illustrates yet another embodiment of the invention in which liquid petroleum gas is imported from an off-site location and introduced into dehydrogenation unit 50 for co-processing with the LPG fraction recovered from unit 40 and the Fischer-Tropsch LPG fraction recovered from fractionator 44. The embodiment of the invention shown in FIG. 3 could be used with stranded reserves providing even less than 100 Mmcf/d but which may provide feasible economics due to the ability to supplement the stranded gas reserve with imported natural gas.

In another embodiment of the invention, the processes depicted in the foregoing embodiments are modularized such that a single processing plant may be alternately configured to process various components of a stranded gas stream as well as to alternately process such stream into various products and product slates. For example, a synthesis gas module may include a gas sweetening/liquids separation unit for removal of certain contaminants, such as sulfur, and separation of liquids from gaseous hydrocarbon components. Such synthesis gas module would generally also include an autothermal reactor for conversion of the gaseous hydrocarbons into synthesis gas. The synthesis gas module may also include one or more Fischer-Tropsch reactors. Alternatively, the Fischer-Tropsch reactor(s) may be combined with one or more Fischer-Tropsch product fractionators to form a Fischer-Tropsch module. One or more product modules may be connected to the synthesis gas module or Fischer-Tropsch module for upgrading the product of the Fischer-Tropsch synthesis into one or more higher value products. One example product module, a transportation fuel product module, would include dehydration or hydrotreatment units for the processing of an LFTL fraction as well as a hydrocracking unit for the processing of an HFTL fraction to obtain a synthetic transportation fuel. Other product modules include, for example, a hydrotreatment plus hydroisomerization unit and a dehydrogenation plus oligomerization units. In some embodiments of the invention, off-site or imported natural gas feed may be piped directly into a product unit.

In yet another embodiment of the invention, a plant, comprised of modules, for the processing of natural gas reserves, and particularly for stranded gas reserves, is provided. In such embodiment, the modules may be land-based or marine-based but in all instances, the modules used are transportable. For example, U.S. Pat. No. 6,277,894, the disclosure of which is incorporated herein by reference, discloses a variety of platform options for such modularized plants. In one embodiment of the invention, the modularized plant is constructed on a seagoing vessel, such as a barge, such that the plant may be moved to stranded gas reserves in marine locations, including off-shore, intra-coastal waterways, and intra-tidal locations. However, other transportable platforms are included in the scope of the invention, including trailer, truckbed, rail car or platform, or other movable forms on which the modules may be transported or moved from location to location.

Embodiments of the invention also provide one or more of the following advantages:

(1) economically feasible recovery of stranded gas reserves having 100 Mmcf/d of natural gas;

(2) transportable module format permitting product slate adaptation depending upon market factors and factors related to the location of the gas reserve; and

(3) modular plant design permitting product slate adaptation.

1. A method of economically and efficiently converting natural gas into one or more liquid hydrocarbon products comprising the steps of:
(a) building a plant comprising one or more modules for the conversion of natural gas into synthesis gas and the conversion of synthesis gas into heavier hydrocarbons and at least one product module on a transportable platform at a location that facilitates heavy construction;
(b) transporting the plant to a location containing sufficient natural gas reserves for operation.
(c) installing the plant near the natural gas reserve; and
(d) producing intermediate or finished liquid hydrocarbon products.

2. The method of claim 1 that uses a Fischer-Tropsch synthesis process for converting natural gas into liquid hydrocarbons.

3. The method of claim 2 that uses air as an oxidant to produce synthesis gas for the Fischer-Tropsch synthesis process.

4. The method of claim 1 wherein the plant is built on a floating platform.

5. The method of claim 4 wherein the plant is transported by means selected from the group of barge and heavy lift ship.

6. The method of claim 4 wherein the plant is installed by means selected from the group of tension legs, a fixed platform, and beaching on shore.

7. The method of claim 1 wherein the hydrocarbon products are selected from the group of naphtha, kerosene, diesel fuel, and jet fuel.

8. The method of claim 7 further comprising the step of distributing the hydrocarbon products in the region where the plant is installed.
